(19) United States
(12) Patent Application Publication
(10) Pub. No.: US 2006/0287567 A1
(43) Pub. Date: Dec. 21, 2006

(54) Therapeutic magnet band

(75) Inventor: Chao-Rong Hsieh, Kaohsiung City (TW)

Correspondence Address: MCNEES, WALLACE & NURICK LLC, 100 PINE STREET, P.O. BOX 1166, HARRISBURG, PA, 17108-1166, US

(73) Assignee: RONG HAN ENTERPRISE CO., LTD., Kaohsiung City (TW)

(21) Appl. No.: 11/253,351

(22) Filed: Oct. 19, 2005

(30) Foreign Application Priority Data
Jun. 20, 2005 (TW) 094210327

(51) Int. Cl. A61N 1/00

(52) U.S. Cl. 600/15

(57) ABSTRACT

A magnet band includes: a flexible band body formed with pockets and through-holes; magnetic elements respectively retained in the pockets of the band body; a pair of belts, each of which has a connecting end portion; and a pair of connectors, each of which includes a pair of tabs secured to the connecting end portion of a respective one of the belts, and each of which is provided with at least one first interlocking member on one of the tabs, and at least one second interlocking member on the other of the tabs. The tabs of each of the connectors are connected to the band body in such a manner that that the first interlocking member on one of the tabs extends through a selected one of the through-holes to interlock the second interlocking member on the other of the tabs.

### CROSS-REFERENCE TO RELATED APPLICATION

This application claims priority of Taiwanese application no. 094210327, filed on Jun. 20, 2005.

### BACKGROUND OF THE INVENTION

1. Field of the invention

This invention relates to a magnet band, more particularly to a magnet band including a band body with magnetic elements, and a pair of belts connected to the band body through a pair of connectors.

2. Description of the related art

FIG. 1 illustrates a conventional magnet belt that includes an elastic belt body 10 with a plurality of magnetic elements 20 mounted thereon. The belt body 10 has two opposite ends provided with fastening means 12, such as hook-and-loop fastening means, so that the belt body 10 can be worn around the user's waist. The magnetic elements 20 can provide therapeutic effects, including pain and stress relief.

U.S. Pat. No. 6,551,234 discloses a magnet belt that includes a supporting portion and a plurality of magnets attached to the supporting portion.

The aforesaid conventional magnet belts are disadvantageous in that their configurations are only suitable for wearing around the user's waist and cannot be adjusted for wearing on the user's back, arms, or legs.

### SUMMARY OF THE INVENTION

Therefore, the object of the present invention is to provide a therapeutic magnet band that is capable of overcoming the aforesaid drawback of the prior art.

According the present invention, there is provided a magnet band that comprises: a flexible band body having two opposite surfaces and formed with a plurality of pockets, and a plurality of through-holes, each of which extends through the surfaces; a plurality of magnetic elements respectively retained in the pockets of the band body; a pair of belts, each of which has a connecting end portion; and a pair of connectors, each of which includes a pair of tabs secured to the connecting end portion of a respective one of the belts, and each of which is provided with at least one first interlocking member on one of the tabs, and at least one second interlocking member on the other of the tabs. The tabs of each of the connectors are detachably connected to the band body in such a manner that the tabs sandwich the band body therebetween and that the first interlocking member on one of the tabs extends through a selected one of the through-holes to interlock releasably the second interlocking member on the other of the tabs.

### BRIEF DESCRIPTION OF THE DRAWINGS

In drawings which illustrate embodiments of the invention,

FIG. 1 is a perspective view of a conventional magnet belt;

FIG. 2 is an exploded perspective view illustrating a state where a pair of belts are to be respectively connected to two short sides of a magnet band of the first preferred embodiment according to this invention;

FIG. 3 is a schematic partly sectional view of the first preferred embodiment;

FIG. 4 is a schematic view to illustrate the first preferred embodiment when worn around the user's waist;

FIG. 5 is an exploded perspective view illustrating a state where the belts are to be respectively connected to two long sides of the band of the first preferred embodiment;

FIG. 6 is a schematic view to illustrate the first preferred embodiment when worn on the user's back;

Fig, 7 is a schematic view to illustrate the first preferred embodiment when worn on the user's back and shoulder;

FIG. 8 is a schematic view to illustrate the first preferred embodiment when worn on the user's thigh;

FIG. 9 is a schematic partly sectional view of the second preferred embodiment of the magnet band according to this invention;

FIG. 10 is a schematic view to illustrate the second preferred embodiment when worn on the user's back; and

FIG. 11 is a schematic view to illustrate the second preferred embodiment when worn on the user's thigh.

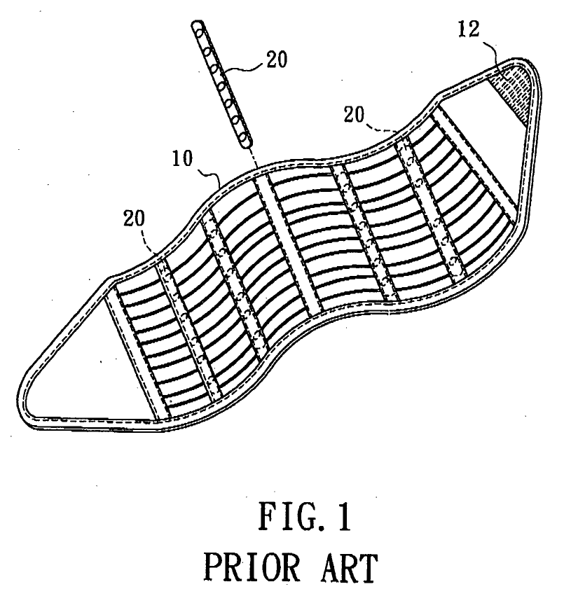

### DETAILED DESCRIPTION OF THE PREFERRED EMBODIMENTS

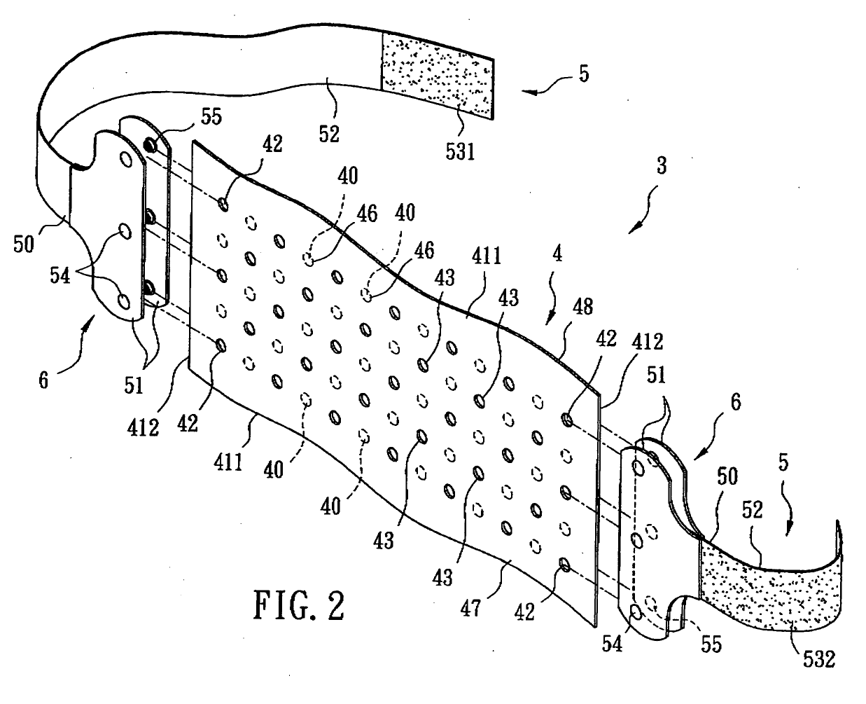

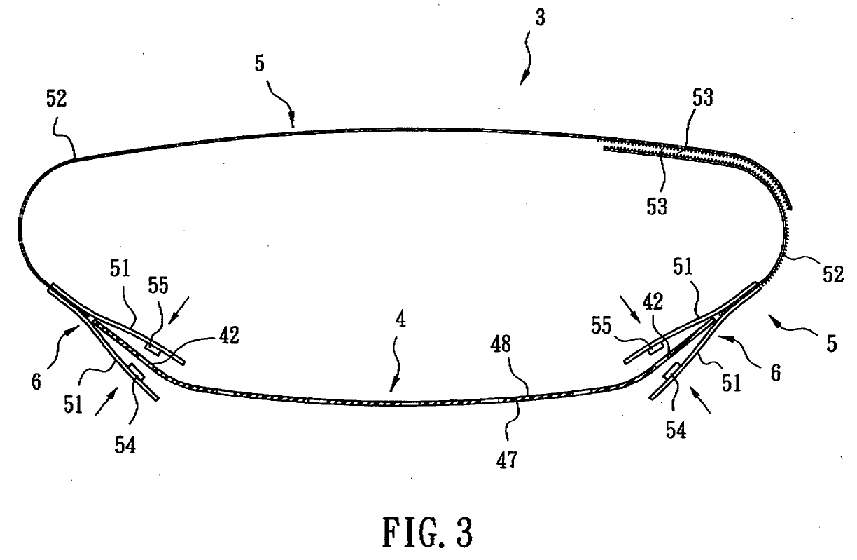

FIGS. 2 and 3 illustrate the first preferred embodiment of a therapeutic magnet band 3 according to the present invention.

The magnet band 3 includes: a flexible band body 4 having two opposite surfaces 47, 48 and formed with a plurality of pockets 46, and a plurality of through-holes 42, each of which extends through the surfaces 47, 48; a plurality of magnetic elements 40 respectively retained in the pockets 46 of the band body 4; a pair of belts 5, each of which has a connecting end portion 50; and a pair of connectors 6, each of which includes a pair of tabs 51 secured to the connecting end portion 50 of a respective one of the belts 5, and each of which is provided with at least one first interlocking member 54 on one of the tabs 51, and at least one second interlocking member 55 on the other of the tabs 51. The tabs 51 of each of the connectors 6 are detachably connected to the band body 4 in such a manner that the tabs 51 sandwich the band body 4 therebetween and that the first interlocking member 54 on one of the tabs 51 extends through a selected one of the through-holes 42 to interlock releasably the second interlocking member 55 on the other of the tabs 51.

In this embodiment, the first interlocking member 54 includes a male button, and the second interlocking member 55 includes a female button. The through-holes 42 are arranged into rows and columns which are respectively disposed along two long sides 411 and two short sides 412 of the band body 4. The tabs 51 of each of the connectors S are provided with a plurality of the first interlocking members 54 that are respectively extendable through a selected one of the rows and columns of the through-holes 42, and a plurality of the second interlocking members 55 that are respectively engageable with the first interlocking members 54.

Each of the belts 5 has a fastening end portion 52 that is opposite to the connecting end portion 50. The fastening end portion 52 of one of the belts 5 is provided with a first fastening member 531. The fastening end portion 52 of the other of the belts is provided with a second fastening member 532 that is engageable with the first fastening member 531 in a hook-and-loop manner. Preferably, the belts 5 are elastic.

Preferably, the band body 4 is further formed with a plurality of apertures 43 for fluid communication between the user's skin, to which the band body 4 is attached, and the ambient.

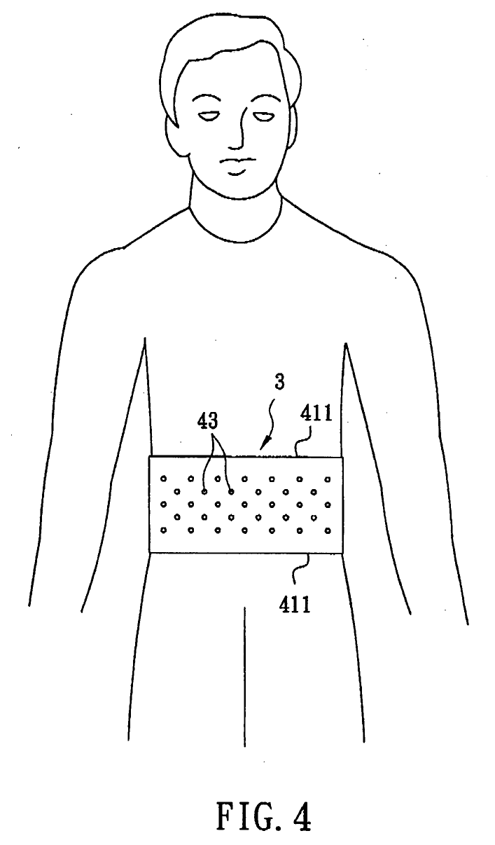

Referring now to FIG. 4, in combination with FIG. 2, when the belts 5 are respectively connected to the short sides 412 of the band body 4 through the connectors 6, the magnet band 3 can be suitably worn around the user's waist.

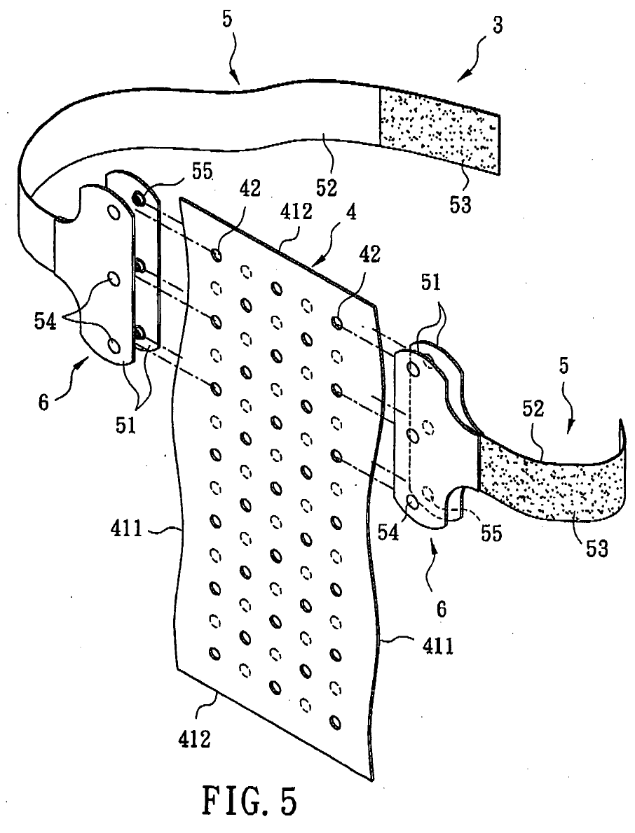

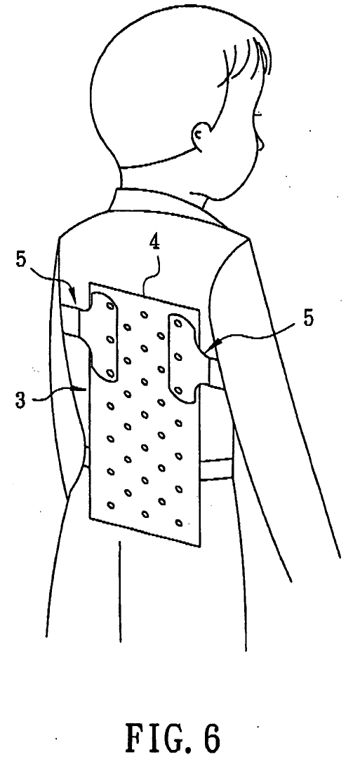

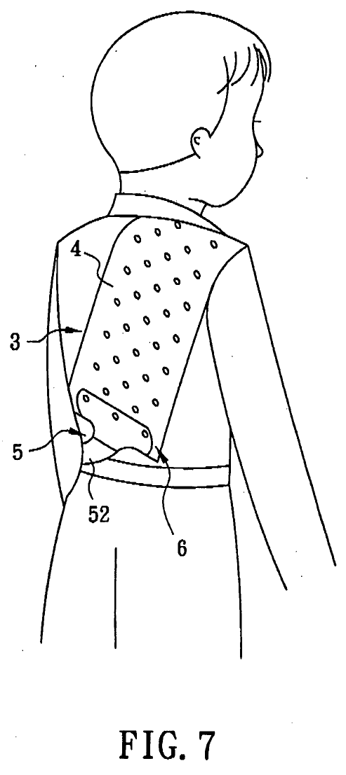

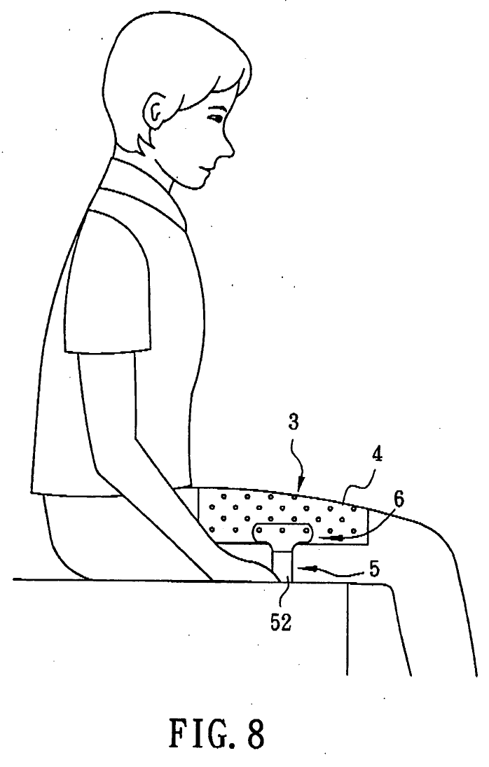

Referring now to FIGS. 5 to 8, in combination with FIG. 2, when the belts 5 are respectively connected to the long sides 411 of the band body 4 through the connectors 6, the magnet band 3 can be suitably worn on the user's back (see FIG. 6), the user's back and shoulder (see FIG. 7), and the user's thigh (see FIG. 8).

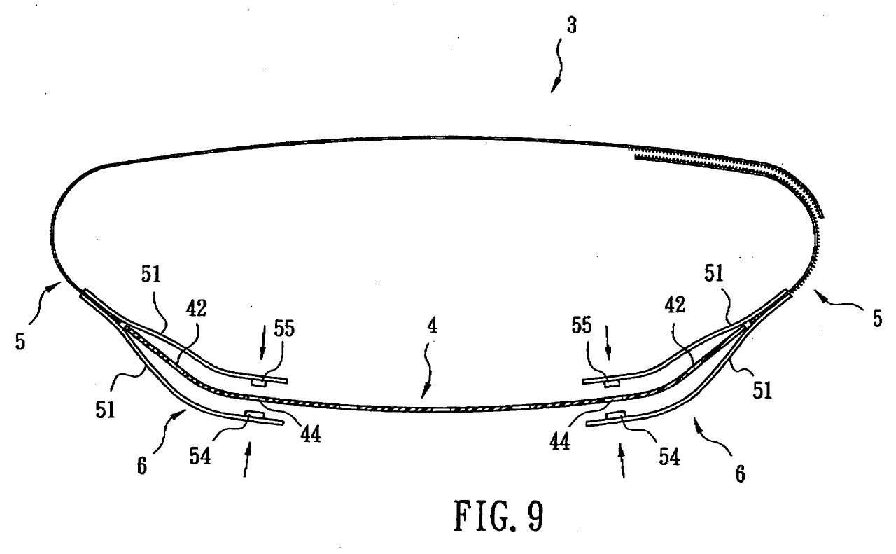

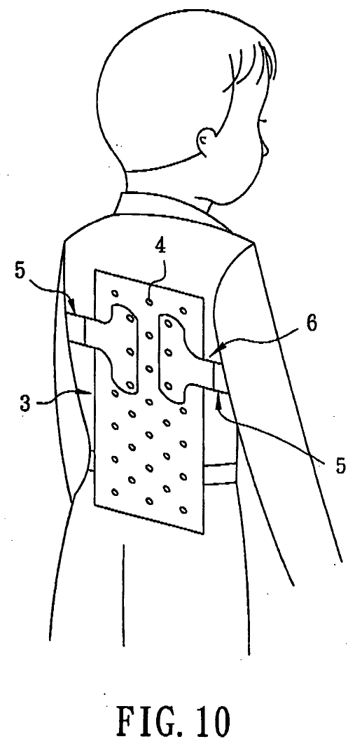

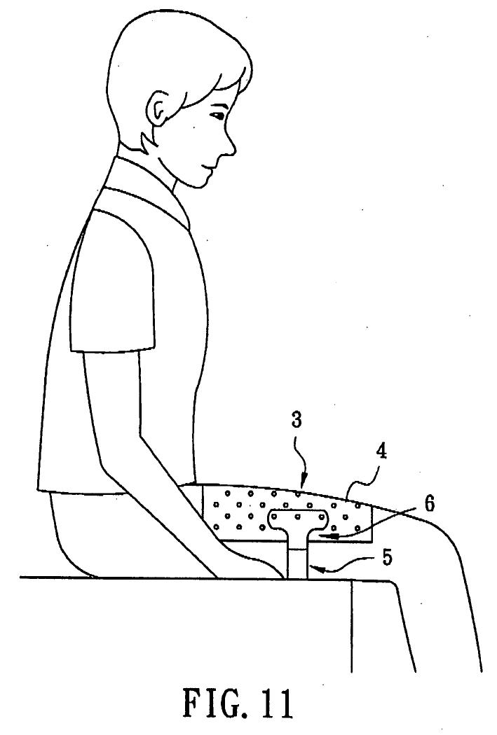

FIG. 9 illustrates the second preferred embodiment of the magnet band 3 according to this invention. The magnet band 3 of this embodiment differs from the previous embodiment in that the band body 4 is further formed with rows and columns of openings 44 for extension of the first interlocking members 54 on the respective one of the tabs 51 of each of the connectors 6 therethrough. The openings 44 in the band body 4 are disposed at an inner portion of the band body 4 and are different from the through-holes 42 which are disposed at a peripheral portion of the band body 4 so as to permit geometrical length adjustment of the belts 5 around the user's body, such as the upper body (see FIG. 10) and the thigh (see FIG. 11) , through selection of the through-holes 42 or the openings 44 for extension of the first interlocking members 54.

With the inclusion of the connectors 6 in the magnet band 3 of this invention, the aforesaid drawback associated with the prior art can be eliminated.

With the invention thus explained, it is apparent that various modifications and variations can be made without departing from the spirit of the present invention.

What is claimed is:

1. A magnet band comprising:
a flexible band body having two opposite surfaces and formed with a plurality of pockets, and a plurality of through-holes, each of which extends through said surfaces;
a plurality of magnetic elements respectively retained in said pockets of said band body;
a pair of belts, each of which has a connecting end portion; and
a pair of connectors, each of which includes a pair of tabs secured to said connecting end portion of a respective one of said belts, and each of which is provided with at least one first interlocking member on one of said tabs, and at least one second interlocking member on the other of said tabs, said tabs of each of said connectors being detachably connected to said band body in such a manner that said tabs sandwich said band body therebetween and that said first interlocking member on said one of said tabs extends through a selected one of said through-holes to interlock releasably said second interlocking member on the other of said tabs

2. The magnet band of claim 1, wherein said first interlocking member includes a male button, and said second interlocking member includes a female button.

3. The magnet band of claim 2, wherein said through-holes are arranged into rows and columns, said tabs of each of said connectors being provided with a plurality of said first interlocking members that are respectively extendable through a selected one of said rows and columns of said through-holes, and a plurality of said second interlocking members that are respectively engageable with said first interlocking members.

4. The magnet band of claim 3, wherein each of said belts has a fastening end portion that is opposite to said connecting end portion, said fastening end portion of one of said belts being provided with a first fastening member, said fastening end portion of the other of said belts being provided with a second fastening member that is engageable with said first fastening member in a hook-and-loop manner.
